(19) United States
(12) Patent Application Publication
(10) Pub. No.: US 2019/0110710 A1
(43) Pub. Date: Apr. 18, 2019

(54) SYSTEMS AND METHODS TO ASSESS INFARCTED MYOCARDIAL TISSUE BY MEASURING ELECTRICAL IMPEDANCE DURING THE CARDIAC CYCLE

(71) Applicants: UNIVERSITAT POLITÉCNICA DE CATALUNYA, BARCELONA (ES); FUNDACIÓ INSTITUT DE RECERCA DE L'HOSPITAL DE LA SANTA CREU I SANT PAU, BARCELONA (ES)

(72) Inventors: Francesc Xavier ROSELL FERRER, BARCELONA (ES); Juan CINCA CUSCULLOLA, BARCELONA (ES); Ramón BRAGÓS BARDIA, BARCELONA (ES); Gerard AMORÓS FIGUERAS, BARCELONA (ES); Esther JORGE VIZUETE, BARCELONA (ES); Tomás GARÍCA SÄNCHEZ, BARCELONA (ES); Benjamin SÁNCHEZ TERRONES, BARCELONA (ES)

(73) Assignees: UNIVERSITAT POLITÉCNICA DE CATALUNYA, BARCELONA (ES); FUNDACIÓ INSTITUT DE RECERCA DE L'HOSPITAL DE LA SANTA CREU I SANT PAU, BARCELONA (ES)

(21) Appl. No.: 16/090,203

(22) PCT Filed: Mar. 30, 2016
(86) PCT No.: PCT/EP2016/056933

§ 371 Date: Sep. 28, 2018

(51) Int. Cl. A61B 5/042; A61B 5/0215; A61B 5/053; A61B 5/00; A61B 5/046

(52) U.S. Cl. CPC A61B 5/0422; A61B 5/0215; A61B 5/02007; A61B 5/6852; A61B 5/046; A61B 5/0538

(57) ABSTRACT

Disclosed herein are methods and devices used to recognize the extent and deepness of infarcted tissue, such as chronic myocardial infarcted tissue. This applies to the heart tissue, but can also be used to assess cicatricial processes in other organs. Examples include injecting pulses of alternating current at a broadband of frequencies while measuring the voltage signal continuously (at a very high sampling rate) to obtain the electrical impedance (Z(f,t)) during the entire cardiac cycle. The impedance measurements may be taken using an intracavitary electrocatheter.

The present disclosure relates to methods and devices to assess infarcted tissue by measuring electrical impedance. More specifically, the methods and devices may be used to recognize the extent and deepness of an infarcted tissue, such as for example myocardial infarcted tissue.

### BACKGROUND

Ventricular arrhythmias are responsible for approximately 60% of sudden deaths in patients with a previous cardiac infarction. Radiofrequency ablation of the arrhythmogenic foci is able to treat around 50-80% of the patients with postinfarction ventricular arrhythmias. The success rate of this procedure could be increased by improvements in the identification and localization of the arrhythmogenic foci in the clinical practice. The clinical intracardiac navigation systems used nowadays (for example: (i) CARTO®, provided by Biosence Webster®, (ii) NavX™, provided by St. Jude Medical™, or (iii) Rhythmia™, provided by Boston Scientific™) locate the postinfarction scar by local measures of voltage using intracavitary electrocatheters. A major drawback of the voltage measurements is that they cannot determine if the scar is completely transmural or not. Another drawback is that these voltage measurements depend on the wave front activation pattern, that can change if the patient suffers an ectopic arrhythmic episode.

The characterization of biological substrates by electrical impedance provides relevant physiological information about the pathological status of the tissues. It has been reported that normal and infarcted myocardium can be recognized by measuring the myocardial electrical impedance (module and phase angle) using an intracardiac electrocatheter. As compared with the normal myocardium, the necrotic infarct scar shows a lower impedance module and a flat phase angle deviation. Measuring the electrical impedance as an indicator of the structural condition of the cardiac tissue has been already proposed in the past. Systems and methods have been described using impedance to identify infarcted regions of heart. However, such systems measure the impedance at a single frequency or at few selected frequencies (between 5 Hz and 50 kHz). With these “old” techniques, only few impedance measurements could be taken during the cardiac cycle due to the time required to inject the wide current spectrum by frequency sweeping. Cardiac movement during contraction and relaxation induces impedance changes that increase the dispersion of impedance measures and this curtails the capacity of the system to recognize the structural derangement.

It is also noted that prompt coronary artery reperfusion in patients with acute myocardial infarction favors cell survival and ultimately promotes the development of heterogeneous transmural infarct scar. The interspaced islands of surviving myocytes may act as slow conducting pathways thereby favoring re-entrant arrhythmias and increasing mortality. Postinfarction ventricular arrhythmias can be suppressed by electrical catheter ablation of the arrhythmogenic substrate but this procedure requires an accurate delineation of the infarct scar and a precise location of the target ablation sites scattered within the infarcted region. The cardiac navigation systems employed in the catheter ablation procedures utilize the mapping of low voltage endocardial electrograms to delineate the borders of the infarct scar although this technique does not allow appropriate discrimination among sites with different degrees of transmural involvement.

In clinical practice, the heterogeneous nature of the infarct scar may only be assessed accurately by cardiac magnetic resonance imaging, but previous studies have reported differential biophysical electrical characteristics between the normal myocardium and the infarcted tissue. Myocardial electrical impedance is a biophysical property of the heart that is influenced by the intrinsic structural characteristics of the myocardial tissue as denoted by experimental models of acute and chronic myocardial infarction. A refinement of the impedance measurement technique was demonstrated by applying fast broadband electrical impedance spectroscopy (EIS) that permitted characterization of the changes in myocardial impedance during the cardiac cycle in normal and acute ischemic conditions in the in situ porcine heart.

### SUMMARY

The present developments may be directed to providing a measuring device for medical applications, which can be used to characterize tissues, for example myocardial tissue structure integrity and solve at least partly the drawbacks and limitations of known systems used in clinical practice. This may be achieved by measuring the changes in impedance during the entire cardiac cycle by injecting electrical current with a broadband spectrum.

A further capability hereof_may be to analyze the changes in myocardial impedance during the cardiac cycle in an infarct scar to detect heterogeneous transmural involvement in the infarcted region.

The present subject matter may take benefit of the measurements of electrical resistivity of heart tissue using the novel technique of fast broadband electrical impedance spectroscopy (EIS). This new technique enables time-varying bioimpedance measurements within the entire cardiac cycle, at simultaneous multiple frequencies (between 1 kHz-1 MHz), obtaining up to 1,000 spectrum measures/sec. With this new procedure it is possible to record the phasic systolic and diastolic changes in myocardial impedance elicited during the cardiac cycle so the movement-induced impedance changes become useful and give additional information. This may increase the accuracy of the technique because it provides more information about the tissue characteristics.

This new system and method may be used to recognize the extent and deepness of infarcted tissue by measuring electrical impedance, for example the extent and deepness of chronic myocardial infarcted tissue by measuring the myocardial electrical impedance during the entire cardiac cycle using one or more intracavitary electrocatheters.

The system and method may be used to assess the extent and deepness of chronic myocardial infarcted tissue by measuring systolic and diastolic myocardial electrical impedance.

The subject matter hereof has been applied specifically to the heart tissue, but can also be used to assess fibrotic processes in other organs.

In a first aspect a method of assessing a cardiac tissue is disclosed. The method may include selecting an area of interest of the cardiac tissue; identifying one or more measurement locations in the selected area of interest; placing an electrocatheter probe at the one or more measurement locations; providing a broadband spectrum signal to the one or more measurement locations using the electrocatheter probe; identifying a diastolic phase of the cardiac cycle; measuring impedance of the cardiac tissue during the identified diastolic phase; identifying a systolic phase of the cardiac cycle; measuring impedance of the cardiac tissue during the identified systolic phase; and assessing said cardiac tissue based on said diastolic and systolic impedance measurements.

Implementations of the methods hereof may include injecting current pulses with a broadband spectrum while measuring the voltage signal continuously (at a sampling rate higher than the maximum spectral component and in the order of ten to a few hundred pulses during a cardiac cycle) to obtain the electrical impedance (Z(f,t)) during the entire cardiac cycle. According to examples hereof, the impedance measurements may be taken using an intracavitary electrocatheter with at least one electrode placed at the tip of the catheter and a skin electrode or another electrocatheter inside the body.

In another aspect, a device is disclosed. The device may include apparatus for selecting an area of interest of a cardiac tissue; apparatus for identifying one or more measurement locations in the selected area of interest; apparatus for placing an electrocatheter probe at the one or more measurement locations; apparatus for providing a broadband signal to the one or more measurement locations using the electrocatheter probe; apparatus for identifying a diastolic phase of the cardiac cycle; apparatus for measuring impedance of the myocardial tissue during the identified diastolic phase; apparatus for identifying a systolic phase of the cardiac cycle; apparatus for measuring impedance of the cardiac tissue during the identified systolic phase; and a system for assessing said cardiac tissue based on said diastolic and systolic impedance measurements.

Another aspect hereof relates to a device. The device may include an arbitrary waveform generator (AWG) to deliver one or more broadband frequency current signals having an amplitude and a duration lasting over a time period associated with one or more cardiac cycles. The device may further have a multielectrode probe, coupled to the AWG, configured to apply the broadband frequency current signals in vivo to a cardiac tissue and measure impedance of the cardiac tissue. The device may further have an acquisition module (AM). The AM may include an electrocardiograph (ECG) recorder and may have a blood pressure recorder. The device may further have a controller coupled to the AWG and to the AM and configured to receive the impedance measurements during the duration of the broadband signal, to receive the recordings of the acquisition module during the duration of the broadband signal, to identify a systolic or diastolic phase of a cardiac cycle, to correlate the impedance measurements with the identified phase of the cardiac cycle, and to identify the cardiac tissue as transmural or non-transmural in response to said correlation.

Implementations hereof may relate to a device and method for mapping the inner (endocardial) regions of the heart, for example an atrial region or a ventricular region, to delineate the extent of necrotic scar. This may be done by measuring the electrical impedance during the entire cardiac cycle after injecting current pulses at multiple frequencies simultaneously. This has a clinical application in the catheter ablation treatment of ventricular arrhythmias in patients with myocardial infarction.

Some advantages of the equipment according to implementations hereof may be:
Unlike other impedance mapping techniques, the present subject matter is based on simultaneous impedance measurements performed at multiple frequencies, e.g. using electrical impedance spectroscopy (EIS), providing more information about the structural condition of the myocardium tissue. Additionally, these simultaneous multifrequency measurements may be performed in a relatively short time compared to the cardiac cycle (in the range of 1 ms, for example between 0.1 and 10 ms, or for example between 0.5 and 2 ms) thus allowing acquisition of the whole time-frequency information in the cardiac cycle. In this way, the impedance spectrum measurements may be performed at known moments of the cardiac cycle eliminating the influence of the myocardium movement. This new device and method may thus allow a more accurate recognition of the extent and transmurality of the infarct scar and permit a better identification of the target sites for electrical ablation of ventricular arrhythmias.
Unlike voltage mapping, impedance mapping is not influenced by the direction of the activation wave front and because of that, it does not require reassessment of the map data whenever an ectopic rhythm supervened during the clinical procedure.
Unlike voltage mapping, impedance mapping can detect the subendocardial, subepicardial and midmyocardial degree of fibrosis. Therefore impedance mapping can detect if fibrosis is transmural or non-transmural.

In yet a further aspect, a system is disclosed. The system may include a device according to one or more other aspects disclosed herein, and an external processing apparatus. The external processing apparatus is connectable to the device via a communication link. The external processing apparatus is configured to run an application to determine the suitable waveform to be uploaded in the AWG, the acquisition strategy, and to apply the algorithms to obtain values of the tissue state estimators from the time-frequency characteristics of the measured impedance signals.

In yet a further aspect, a controller is disclosed. The controller may include a signal selector module, configured to be coupled to an arbitrary waveform generator (AWG), to provide to the AWG parameters of a broadband current pulse signal to be applied on a cardiac tissue. The controller may further include a receiver configured to be coupled to an acquisition module to receive impedance measurements and ECG and blood pressure recordings from the acquisition module. The controller may further include a processing module, configured to identify a systolic and/or diastolic phase of the cardiac cycle as a function of said received recordings. Furthermore, the controller may include an assessment module, configured to identify a state of the cardiac tissue as a function of the impedance measurements and the identified phase.

In another aspect, a computer program product is disclosed. The computer program product may include program instructions for causing a computing system to perform a method of assessing cardiac tissue according to some examples disclosed herein. The computer program may merge the results obtained with the impedance map with the results obtained with the voltage mapping.

The computer program product may be embodied on a storage medium (for example, a CD-ROM, a DVD, a USB drive, on a computer memory or on a read-only memory) or carried on a carrier signal (for example, on an electrical or optical carrier signal).

The computer program may be in the form of source code, object code, a code intermediate source and object code such as in partially compiled form, or in any other form suitable for use in the implementation of the processes. The carrier may be any entity or device capable of carrying the computer program.

For example, the carrier may be or include a storage medium, such as a ROM, for example a CD ROM or a semiconductor ROM, or a magnetic recording medium, for example a hard disk. Further, the carrier may be a transmissible carrier such as an electrical or optical signal, which may be conveyed via electrical or optical cable or by radio or other methods, devices or systems.

When the computer program is embodied in a signal that may be conveyed directly by a cable or other device or method or system, the carrier may be constituted by such cable or other device or method or system.

Alternatively, the carrier may be an integrated circuit in which the computer program is embedded, the integrated circuit being adapted for performing, or for use in the performance of, the relevant methods.

### BRIEF DESCRIPTION OF THE DRAWINGS

Particular implementations of the present subject matter will be described in the following by way of non-limiting examples, with reference to the appended drawings, in which:

FIG. 1A is a schematic representation of the system used to record simultaneously the phasic changes of myocardial electrical impedance, left ventricular (LV) pressure and surface ECG;

FIG. 1B schematically illustrates various example electrocatheter configurations;

FIG. 2 is a representation of the (non-)periodic broadband EIS for the nonparametric-in-time measurement of the time-varying bioimpedance;

FIG. 3 displays an example of a multisine signal and its spectrum;

FIG. 4A represents a resistivity spectrum which has a modulation due to myocardium movement;

FIG. 4B represents the modulation of the resistivity waveform due to myocardium movement;

FIG. 5 is a Wessel plane showing a set of arcs corresponding to several cardiac cycles;

FIG. 6A represents the time course of R0, R∞ obtained by acquiring 60 spectra per second and fitting them to the Cole model for impedance;

FIG. 6B represents the time course of α obtained by acquiring 60 spectra per second and fitting them to the Cole model for impedance;

FIG. 6C represents the time course of fc obtained by acquiring 60 spectra per second and fitting them to the Cole model for impedance;

FIG. 7 is a flow diagram of a method of assessing a cardiac tissue;

FIG. 7A displays impedance spectra of three regions with three tissue states and their modulation range due to myocardium movement in the three tissue states;

FIG. 7B shows the arcs in the Wessel plane for the three regions.

FIG. 8 shows the time-domain of impedance magnitude at a selected frequency (1 kHz) of 4 different tissues states;

FIG. 9 shows magnitude of the normal (NZ), border (BZ) and healed-scar (infarcted, IZ) zones impedance time signal at three different frequencies (1 kHz, 307 kHz and 939 kHz);

FIG. 10 shows the corresponding phase angle at the same frequencies as the ones indicated in FIG. 9;

FIG. 11 represents the resistivity time course at a frequency of 1 kHz, for two tissue states (normal and acute ischemic) together with the synchronously acquired ventricle pressure, its derivative and the ECG;

FIG. 12A represents the resistivity at a given frequency (top) and the left ventricular pressure (LVP) (bottom);

FIG. 12B shows the cycle described by the evolution of the signal in the Resistivity-LVP plane;

FIG. 13A shows the Resistivity-LVP loop at baseline and after 30 min. of left anterior descending (LAD) coronary artery occlusion;

FIG. 13B shows the Resistivity-LVP loop area mean at different frequencies at baseline and after 30 minutes of ischemia;

FIG. 14 shows the Resistivity-LVP loop for four different tissue-states;

FIG. 15 illustrates a schematic representation of a heart with two electrocatheters;

FIG. 16A illustrates the mean value of resistivity (top) and phase angle (bottom) vs. the excitation frequency for three different tissues: healthy tissue (N), non-transmural and transmural infarcted scar (ISN and IST, respectively);

FIG. 16B illustrates the mean values of resistivity (top) and phase angle (bottom) at 4 selected frequencies (1, 41, 307 and 1000 kHz) for the three tissue;

FIG. 17 illustrates a linear correlation between myocardial resistivity (upper-left)/phase angle (lower-left) and the percentage of fibrosis;

FIG. 18 illustrates the comparative pattern of the Cole impedance model obtained in normal (N: white), non-transmural scar (ISN: grey) and infarcted transmural scar (IST: black);

FIG. 19A illustrates myocardial tissue electrical resistivity and ECG in a normal myocardium;

FIG. 19B illustrates myocardial tissue electrical resistivity and ECG in a non-transmural infarct scar;

FIG. 19C illustrates myocardial tissue electrical resistivity and ECG in a transmural infarct scar;

FIG. 20 is a multiparametric analysis showing that a combination of resistivity and fc improve the predictive ability to discriminate between both fibrotic tissues.

### DETAILED DESCRIPTION OF EXAMPLES

Non-limiting examples of the present disclosure will be described in the following, with reference to the appended drawings.

Examples of the present subject matter provide a system of monitoring myocardial tissue that may include:
At least one contact electrode on the body; and
At least one electrode placed at the tip of an electrocatheter to be inserted through blood vessels or body openings.

The steps to perform an impedance measurement using this system may include:
1—Generate and apply to the patient an alternating broadband electrical current signal through the electrodes of an intracavitary electrocatheter, between an electrode of an intracavitary electrocatheter and an external skin electrode or between electrodes placed in two different electrocatheters.
2—Measure the voltage signals across a given pair of electrodes of the electrocatheter and/or between an electrode of an intracavitary electrocatheter and an external skin electrode.
3—Determine the impedance at each frequency and fit the values to a custom mathematical model.
4—The fitted parameters are the inputs of an algorithm that outputs a numerical value directly related to the tissue structural integrity. This algorithm may take into account:
   The values and the absolute or relative changes in impedance or admittance magnitude or phase angle (or alternative representations as real and imaginary part of impedance or admittance, or as intrinsic parameters, resistivity, conductivity and permittivity) or in the impedance or admittance model parameters in selected points of the cardiac cycle. In a preferred case, in the systolic and diastolic points determined by synchronism with other physiological signals (ECG, arterial or left ventricular pressure), or in the maximum and minimum of impedance or admittance related signals.
   The shape (slope in selected points, number and type of local maxima and minima, spectral content) of impedance magnitude or phase angle, or their alternative representations as real and imaginary parts, or in the signals corresponding to the time evolution of the impedance model parameters.
   The ratios, differences, areas determined by the aforementioned signals at different frequencies or model parameters or the combination between them and other physiological signals (ECG, arterial or left ventricular pressure).

Apparatus and Method Description

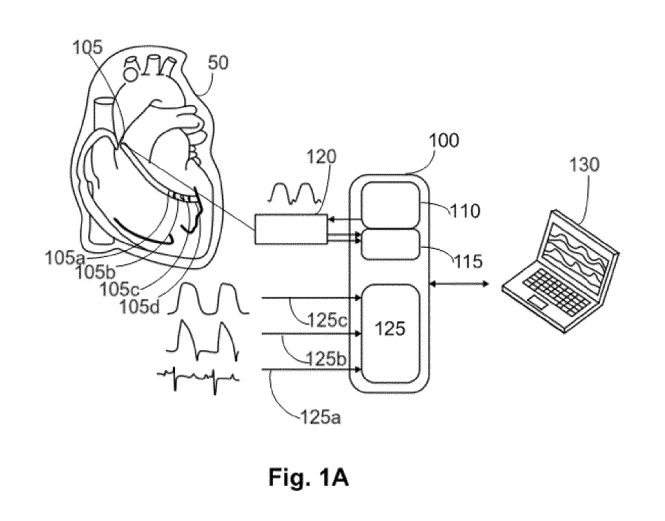

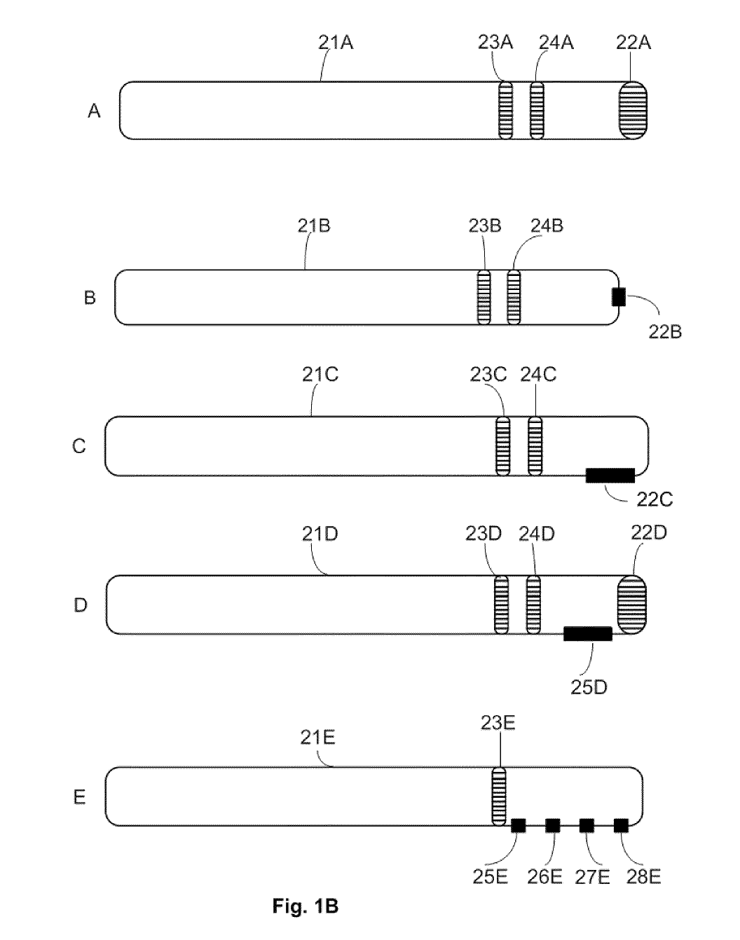

FIG. 1A is a schematic representation of the system used to record simultaneously the phasic changes of myocardial electrical impedance, left ventricular (LV) pressure and surface ECG. Electrical impedance at a myocardium 50 was measured, using a four electrode electrocatheter 105, by applying an alternating current between the outer pair of electrodes (105a, 105d), while the inner two electrodes (105b, 105c) were employed to measure the resulting potential difference. AFE: analog front-end.

The apparatus is made up of the following blocks (FIG. 1A):
1. A main block that includes an arbitrary waveform generator 110 (AWG-Signal generator) that generates the waveform of the signal to be injected to the myocardium and a digitizer 125 that acquires the signals coming from the front-end (AFE). It also takes care of the synchronism between generation and acquisition. The main block includes a processor to control the sequence of operations.
2. A front-end block 120 that adapts the signal from the AWG-Signal generator 110 to a voltage or current range that fits the electrical safety standards and minimizes the effect of the electrode-tissue impedance. It also includes one or several voltage detection channels to amplify the voltage signals in the catheter and skin electrodes also minimizing the effect of the electrode-tissue impedance. It can also detect and amplify differences between these voltages. A channel to measure the injected current can also be included. The front-end can be adapted to several electrode configurations (2, 3 and 4 electrode configurations) and even switch between them. In cases where more than four electrodes (including skin or electrocatheter electrodes) are used, the frond-end can also select the most appropriate electrodes to inject or detect the signals.
3. A set of electrodes (105a, 105b, 105c, 105d) placed in the tip of a catheter and in the surface of the subject. Preferred electrode combinations can be, for example:
   2 electrodes: one in the catheter tip and one external, in the subject surface.
   2 electrodes: one in the catheter tip and one annular near the catheter tip.
   3 electrodes: one in the catheter tip, one annular near the catheter tip and one external, in the subject surface.
   4 electrodes: one in the catheter tip, one annular near the catheter tip and two external, in the subject surface.
   2, 3 or 4 electrode technique using two different catheters in different locations of the heart.
4. Additional channels or connection to additional measurement systems if integrated in a higher level apparatus, to acquire ECG, pressure and/or flux signals synchronously with the impedance acquisitions.
5. A computer or external processor 130 connected to the apparatus 100 controller through a communication link, running an application that determines the suitable waveform to be uploaded in the AWG 110, the acquisition strategy, that applies the algorithms to obtain the values of the tissue state estimators from the time-frequency characteristics of the measured impedance signals and creates and displays a map of tissue properties that could be merged with the voltage mapping.

FIG. 1B schematically illustrates various electrocatheter configurations A-E that may be used alternatively. Electrocatheter A includes a body 21A, an electrode 22A and ring electrodes 23A and 24A. Electrode 22A may be used for measuring impedance and/or for ablation purposes. The ring electrodes may be used to detect voltage or to inject current. Electrocatheter B may include a body 21B, an electrode 22B and ring electrodes 23B and 24B. Electrode 22A may be smaller than the one used in electrocatheter A and may be used for measuring voltage or for injecting current. The ring electrodes may be used, as previously, to detect voltage or to inject current. Electrocatheter C may be similar to electrocatheter B. It may have a body 21C, an electrode 22C and ring electrodes 23C and 24C. However, electrode 22C may be to a lateral position and not at the tip of body 21C and may also be used for measuring impedance or for injecting current. The ring electrodes may be used, as previously, to detect impedance or to inject current. Electrocatheter D may have a body 21D, an electrode 22D at the tip of the body 21D, ring electrodes 23D and 24D and a lateral electrode 25D. Electrode 22D may be used for ablation whereas electrode 25D may be used for measuring voltage or for injecting current. The ring electrodes may be used, as previously, to detect voltage or to inject current. Electrocatheter E may have a body 21E, one or more ring electrodes 23E and an array of four lateral electrodes 25E, 26E, 27E and 28E. The lateral electrodes may be used for injection or voltage detection and measurement whereas the ring electrodes may be used, as previously, to detect voltage or to inject current, accordingly. Alternatively, the array of four lateral electrodes may include a mechanism to separate them from the catheter surface so that they may contact the myocardium tissue without the catheter body coming in contact with the tissue. In another alternative implementation, the array of four electrodes may be arranged around the tip of the catheter. Another alternative is to use a catheter with many electrodes, for example over an almost cylindrical surface that may be inflated against the surface of a cardiac chamber and to take measurements between selectable electrodes.

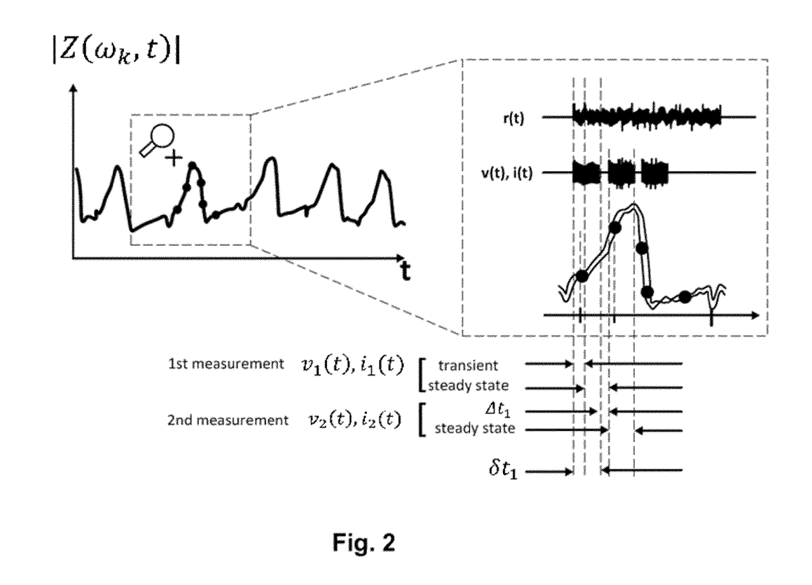

Instead of using a sequence of sinusoidal signals, whose frequency is swept then acquiring information about the impedance at different frequencies in different parts of the cardiac cycle, the fast broadband EIS methods inject bursts or a continuous periodic signal that contains a set of measurement frequencies simultaneously. The minimum length of this signal is one period of the slowest frequency. That is, in a typical case, 1 ms for a minimum frequency of 1 kHz. This would allow acquiring a maximum of 1000 whole impedance spectra per second. Nevertheless, this is usually not needed and a few tenths of spectra per second are enough. The most important is, however, the fact that the acquisition of the resulting voltage and current signals only takes 1 ms, then acquiring a quasi-static picture of the tissue state in a given point of the cardiac cycle. FIG. 2 describes this acquisition method. The voltage and current bursts are drawn longer that they can be for clarity. They can be as short as 1/1000^{th} of the approximate cycle period.

FIG. 2 is a representation of the (non-)periodic broadband EIS for the nonparametric-in-time measurement of the time-varying bioimpedance Z(ωk,t), with ωk being the excitation (angular) frequency. Two possible approaches exist, where the bio-system is excited either (A) with a continuous reference broadband signal r(t)or (B) with a non-continuous one.

There is a variety of signals that allow acquiring a whole spectrum in a given bandwidth: filtered noise, pseudo-random pulse sequences, Discrete Interval Binary Sequences, chirp signals and multisine or multitone signals. This last type of signals, which are in the addition of a given number of sinusoidal signals, is preferred for our usage because it applies the minimum amount of energy to the tissue given that they only have energy in the selected frequency samples. In other words, for a given energy limit of the injected signal, due to electrical safety reasons, the components of the multisine can have more amplitude than other signals, then allowing to reach a higher signal to noise ratio and then a higher accuracy in the spectrum estimation at the selected frequencies.

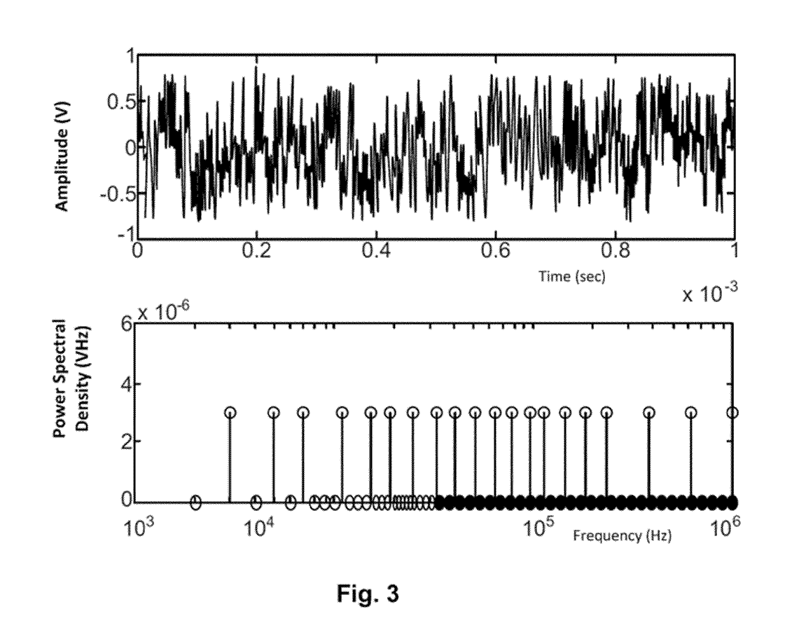

FIG. 3 displays an example of multisine signal and its spectrum. The amplitudes and frequencies can be distributed to reach an optimal adaptation to the expected impedance spectrum and there are several methods to distribute the phase of the multisine components in order to reach a minimum amplitude, for a given RMS signal value given by the amount of frequency components and their corresponding amplitudes. This is usually referred to as obtaining a minimal Crest Factor (the ratio between the peak amplitude and the RMS value. Crest factors in the range of 1.5 to 3 can be obtained for multisines with 20-25 components.

With the described apparatus, method and signal, we are capable of obtaining any time-frequency impedance feature in the 1 kHz-1 MHz range and for the times involved in the dynamic behavior of the beating heart.

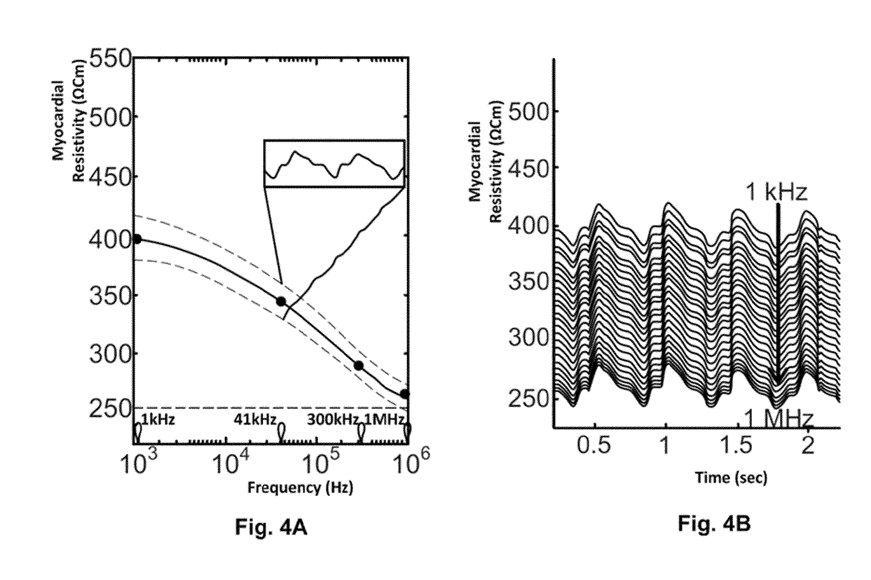

FIG. 4A summarizes this by representing a resistivity spectrum which has a modulation due to myocardium movement, but being now this modulation a source of information, given that the waveform of the impedance as a function of time can be determined at every measured frequency (41 kHz for example in the figure).

FIG. 4B represents the modulation of the resistivity waveform due to myocardium movement. It shows the time course of the impedance as a function of time, represented for all 26 measured frequencies. The mean value clearly changes, but also the signal shapes may change from low frequencies to high frequencies, and may change in a different way if there is a pathology.

If the signals are acquired with enough quality and there is a suitable calibration method to correct the errors induced by electrodes, cables and the acquisition system frequency response, the acquired spectra can be fitted to one or several curves that follow the Cole model and then be parametrized by four parameters. The equation for the impedance representation is the following one:

$$Z ( f ) = {R}_{∞} + \frac{{R}_{0} - {R}_{∞}}{1 + {( j \frac{f}{{f}_{c}} )}^{α}} \qquad Equation 1. Cole model$$

The parameter R₀ provides information on the extracellular space while R_{∞} depends on the total volume and the ratio between them on the cell density. The central relaxation frequency f_{c} depends on the average cell size and the parameter α is related to the cell shape and size homogeneity. That means that structural information about the tissue can be obtained from the spectra obtained at every cycle point. If represented in the Wessel plane, the impedance spectra of impedance relaxations of biological materials describe circumference arcs.

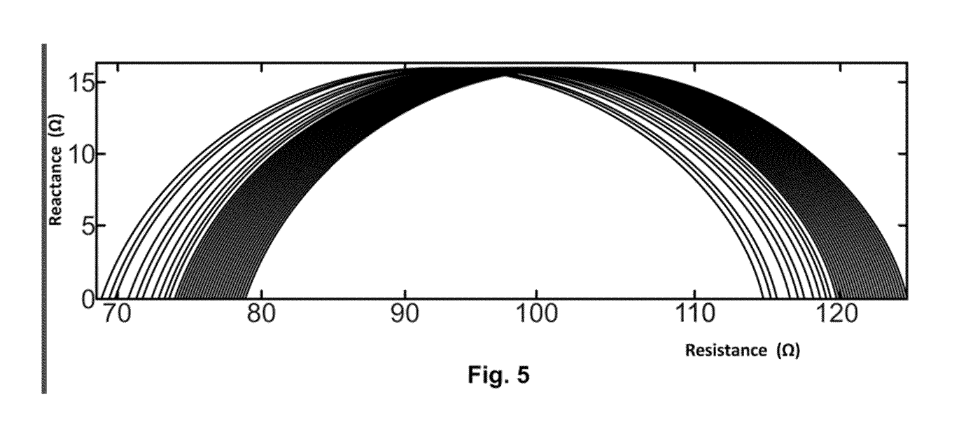

FIG. 5 (Wessel plane) displays the set of arcs corresponding to several cardiac cycles.

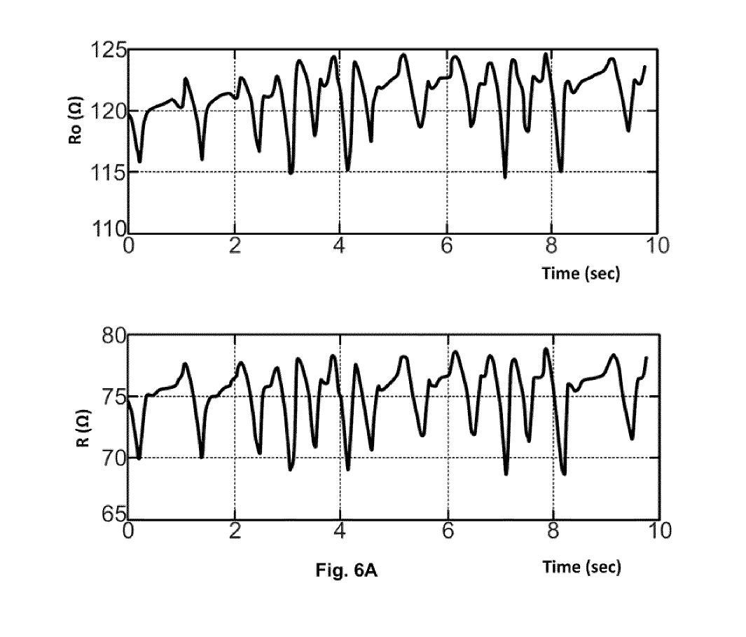

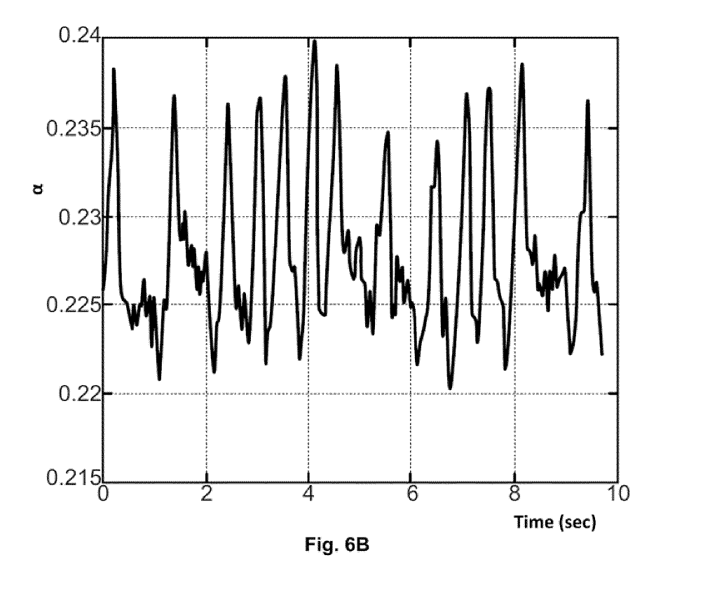

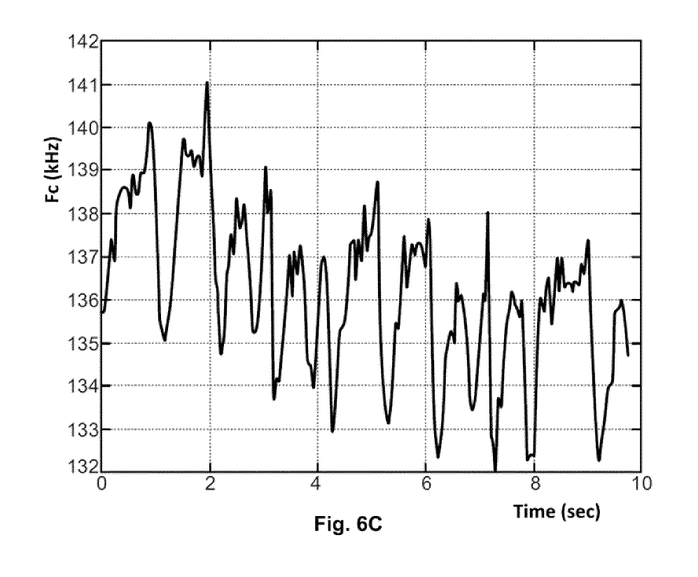

FIG. 6A represents the time course of R₀, R_{∞}, FIG. 6B the time course of α and FIG. 6C the time course of f_{c} obtained by acquiring 60 spectra per second and fitting them to the Cole model for impedance.

Summarizing, with the presented combination of apparatus, acquisition method and signal, it is possible to place a catheter in a given point of the myocardium and, in the time corresponding to a beat cycle, acquire an amount of impedance spectra able to characterize the tissue including time and frequency information, then allowing a better characterization that would help in identifying the tissue structural integrity in endocardial or epicardial mapping procedures. This should improve the detection of the areas with non-transmural infarction, which could be arrhythmia foci, in the catheter ablation treatment of malignant ventricular arrhythmias in patients with myocardial infarction.

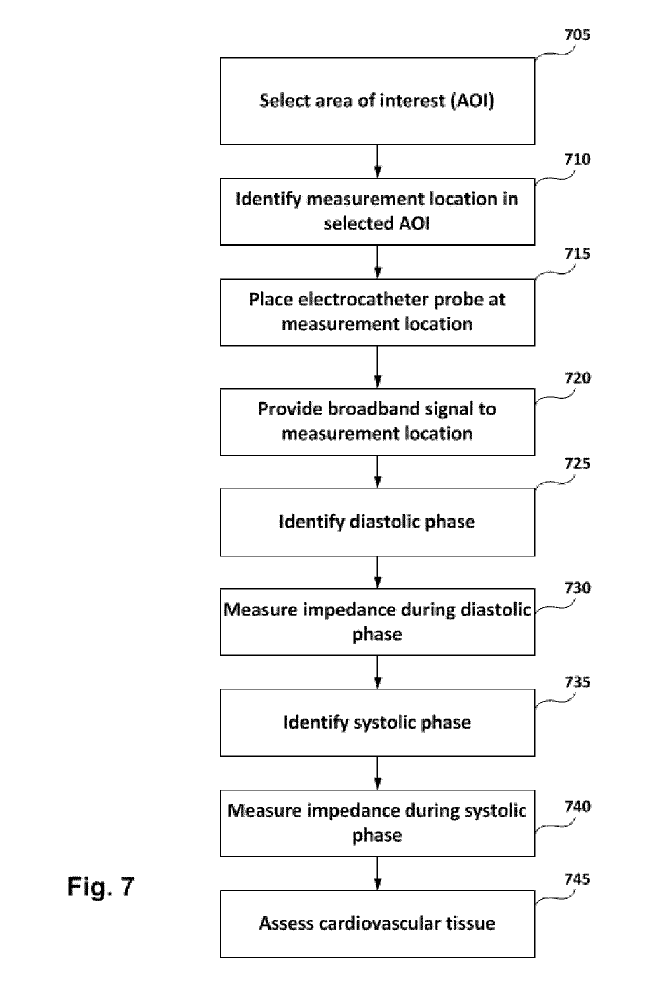

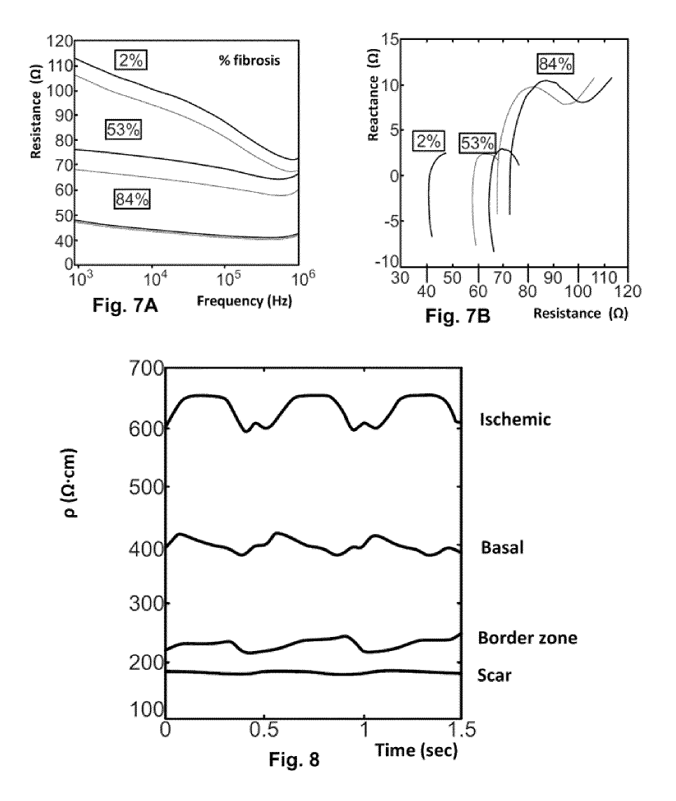

FIG. 7 is a flow diagram of a method of assessing a cardiac tissue. In a first block 705, an area of interest of the cardiac tissue is selected. Then, in block 710, one or more measurement locations in the selected area of interest are identified. In block 715, an electrocatheter probe at the one or more measurement locations is placed. In block 720, a broadband spectrum signal is provided to the one or more measurement locations using the electrocatheter probe. In block 725, a diastolic phase of the cardiac cycle is identified. In block 730 the impedance of the cardiac tissue is measured during the identified diastolic phase. In block 735, a systolic phase of the cardiac cycle is identified. In block 740 impedance of the cardiac tissue is measured during the identified systolic phase. Finally, in block 745, the cardiac tissue is assessed and displayed based on the diastolic and systolic phase impedance measurements.

In the following figures, several examples of signals that can be used to derive estimators of the myocardial state are shown.

FIG. 7A displays impedance spectra of three tissue states and their modulation range due to myocardium movement in three tissue states, characterized by their fibrosis percentage: normal tissue (2% fibrosis), healed scar (84% fibrosis) and border zone (53% fibrosis).

FIG. 7B shows the arcs in the Wessel plane for the three regions. It can be seen how normal tissue has higher impedance while healed scar has not only lower average impedance but also lower modulation and an almost flat spectrum, revealing its resistive behaviour due to the absence of viable cells. Border zone is similar to the non-transmural infarction areas that can be foci of arrhythmia.

FIG. 8 shows the time-domain of impedance magnitude at a selected frequency (1 kHz) of 4 different tissues states. In this case, also an acute ischemic area is included. It can be seen how the different tissue-state areas provide not only different average impedance values, but also different modulations and different signal shapes.

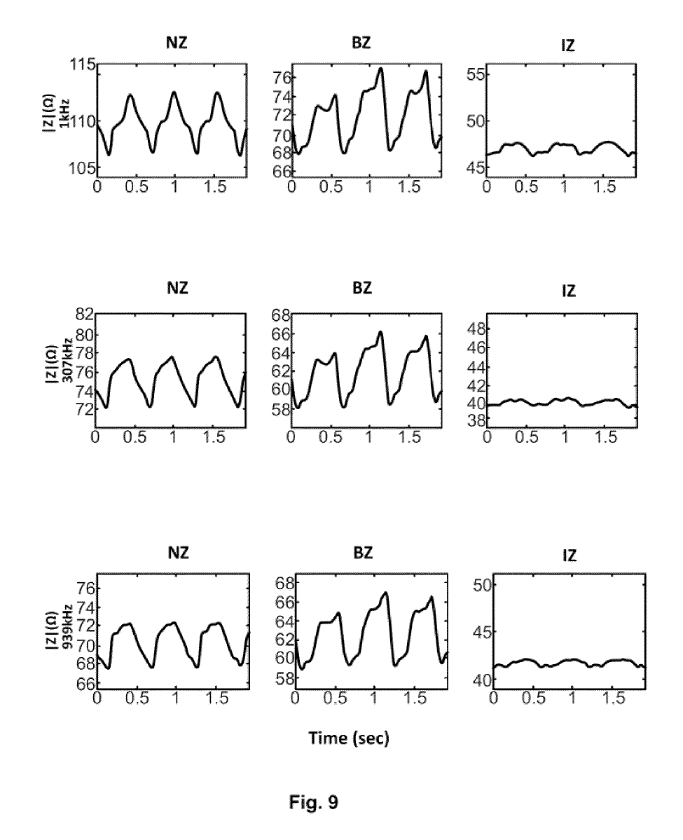

FIG. 9 shows magnitude of the normal (NZ), border (BZ) and healed-scar (infarcted, IZ) zones impedance time signal at three different frequencies (1 kHz, 307 kHz and 939 kHz).

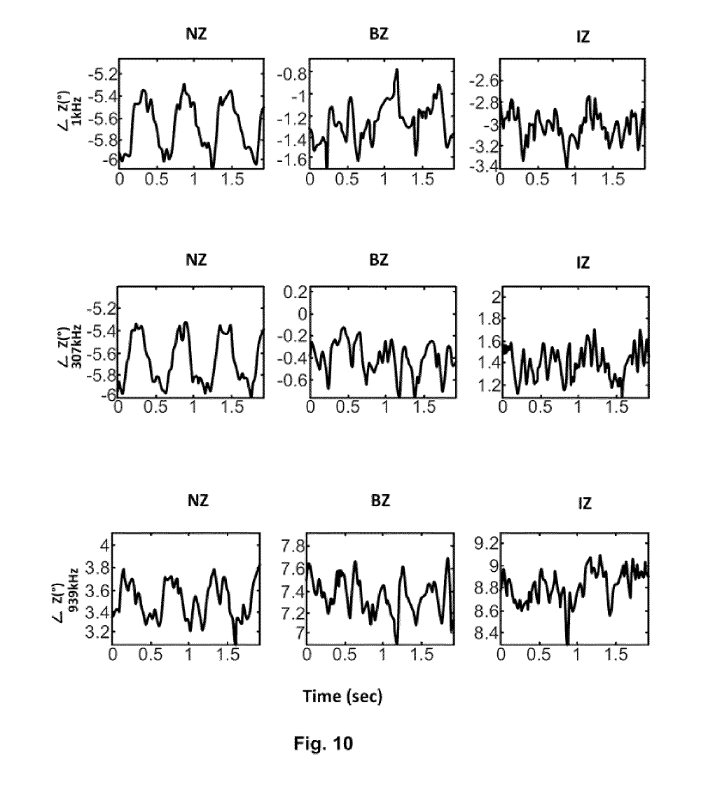

FIG. 10 shows the corresponding phase angle at the same frequencies as the ones indicated in FIG. 9. It can be seen how not only the average value, modulation and signal shape (slope, symmetry, number of local maxima and minima, complexity (spectral content), but also how these features change with frequency. Therefore it is possible to define estimators with more information content and more ability to separate measures using the combined time and frequency content.

Up to the extent of what has been described, the variables involved in the definition of estimators of the tissue state can be not only impedance but also admittance or the related intrinsic parameters, resistivity, conductivity and permittivity. For all of them, the estimators can take into account the values and the absolute or relative changes of the magnitude or phase angle or their alternative representations as real and imaginary part. It can also take into account the model parameters after fitting any of the aforementioned variables spectrum to a mathematical model. Their time course or their values in selected points of the cardiac cycle. In a preferred case, in the systolic and diastolic points determined by synchronism with other physiological signals (ECG, arterial or left ventricular pressure), or in the maximum and minimum of impedance or admittance related signals. Information can also be contained in the delay respect these signals.

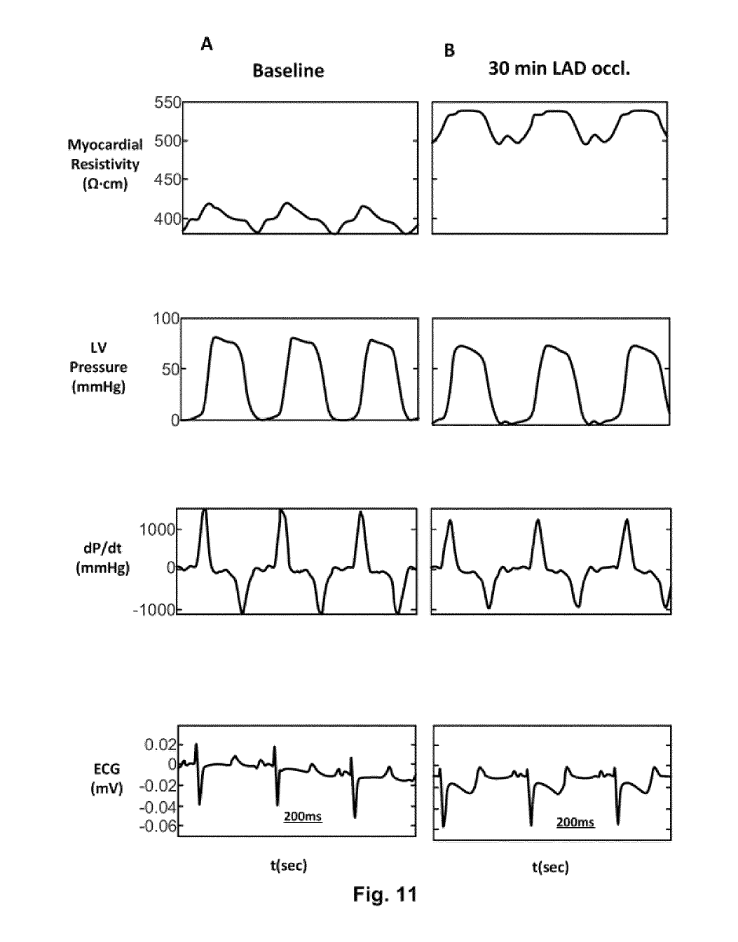

FIG. 11 represents the resistivity time course at a frequency of 1 kHz, for two tissue state (normal and acute ischemic) together with the synchronously acquired ventricle pressure, its derivative and the ECG.

Additionally to the possible definition of pathology-dependent delays between the reference signals (ECG, arterial or left ventricular pressure) and the impedance signals, there is a specific representation that gives information about the work performed by the myocardium section that is being measured, and that, consequently, will change depending on the tissue state.

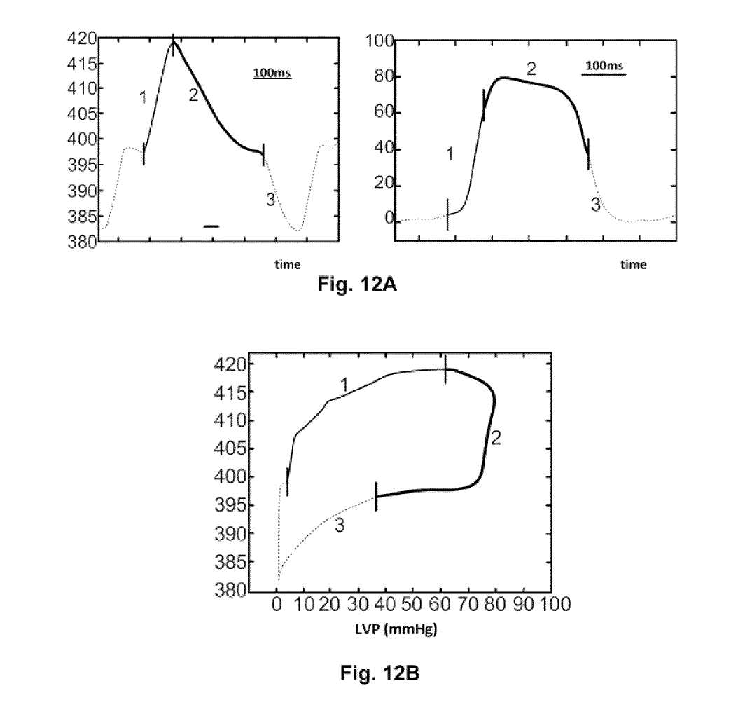

This is represented in FIG. 12A and FIG. 12B. FIG. 12A represents the resistivity at a given frequency (top) and the left ventricular pressure (LVP) (bottom). In FIG. 12B, the cycle described by the evolution of the signal in the Resistivity-LVP plane is shown. This should not be mistaken with the well-known Pressure-Volume diagram, whose area represents the work performed by the whole ventricle. In this case we are representing for the first time a figure whose cycle follows the evolution of a global ventricle variable (the pressure) and the impedance in a localized point. Being the impedance dependent on the myocardium movement in that point, the area of the figure would depend on the work performed by that part of the myocardium. Then it would allow distinguishing active regions (those where the cells contraction provokes the pressure change and where the cells contraction precedes the pressure rise from passive zones, those where tissue deformation is consequence of the pressure change and where both changes are in-phase.

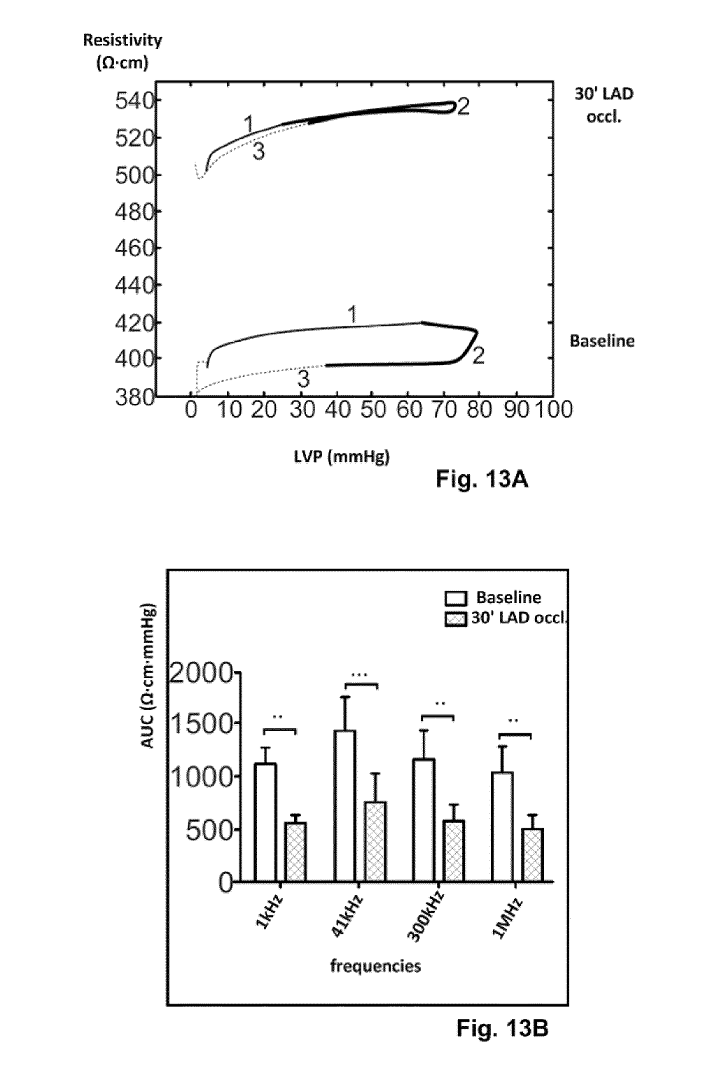

FIGS. 13A and 13B respectively show the Resistivity-LVP loop at baseline and after 30 min. of left anterior descending (LAD) coronary artery occlusion, and the Resistivity-LVP loop area mean at different frequencies at baseline and after 30 minutes of ischemia. The figures show how the area of the Resistivity-LVP loop is reduced after 30 minutes of acute ischemia compared to its baseline value. Both tissues have mobility, but the ischemic one is following passively the movement induced by the pressure, showing the impedance less delay respect to the pressure and being the Resistivity -LVP diagram near to a line, then having less area. FIG. 13B shows how the separation between both tissue states is better at a given frequency, then having again the time-frequency impedance acquisition an added value.

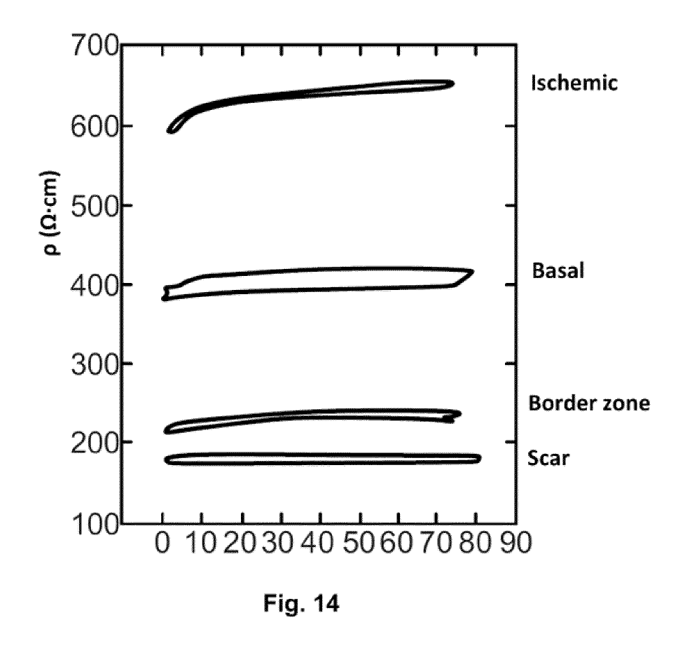

FIG. 14 shows the Resistivity-LVP loop for four different tissue-states. The healed scar not only has lower resistivity but also shows almost no area because it follows the movement passively, then in-phase with the LV pressure. The border-zone, our detection target, has a smaller impedance and impedance modulation than the normal (basal) tissue, but, still having active myocytes, it performs work and shows higher area value.

The obvious fact that the impedance changes are related not only with the tissue state but with the motility can be corroborated by applying drugs that affect motility without destroying the cells.

Myocardial healthy tissue, non-transmural infarct zones and transmural infarct zones can be recognized in vivo by specific changes of their myocardial electrical impedance. Myocardial impedance mapping can identify the degree of fibrosis, and therefore the extent and transmurality of the infarct scar. This technique may improve the yielding of catheter ablation of ventricular arrhythmias in patients with chronic myocardial infarction.

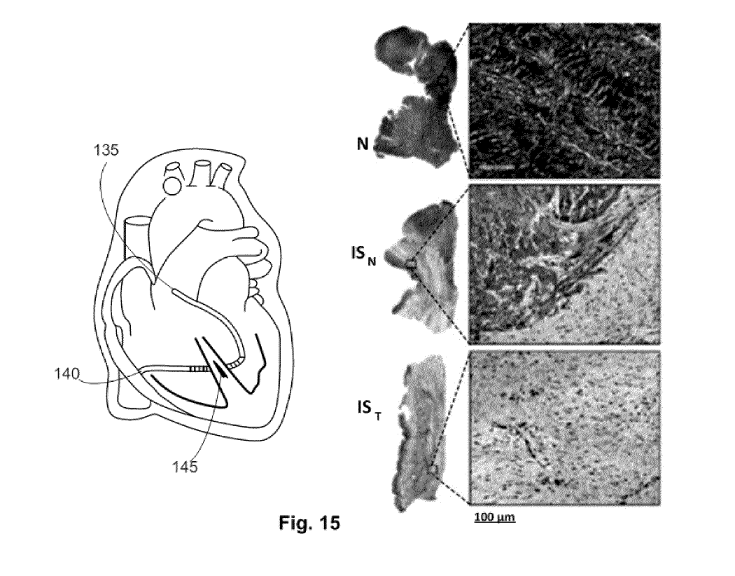

FIG. 15 illustrates a schematic representation of a heart with two electrocatheters; one in the left ventricle 135 and one in the right ventricle 140. The grey area in between the catheters 145 represents an infarct scar in the mid-cardiac septum that could not be detected using the measure of voltage but that can be detected by the impedance spectroscopy measurements.

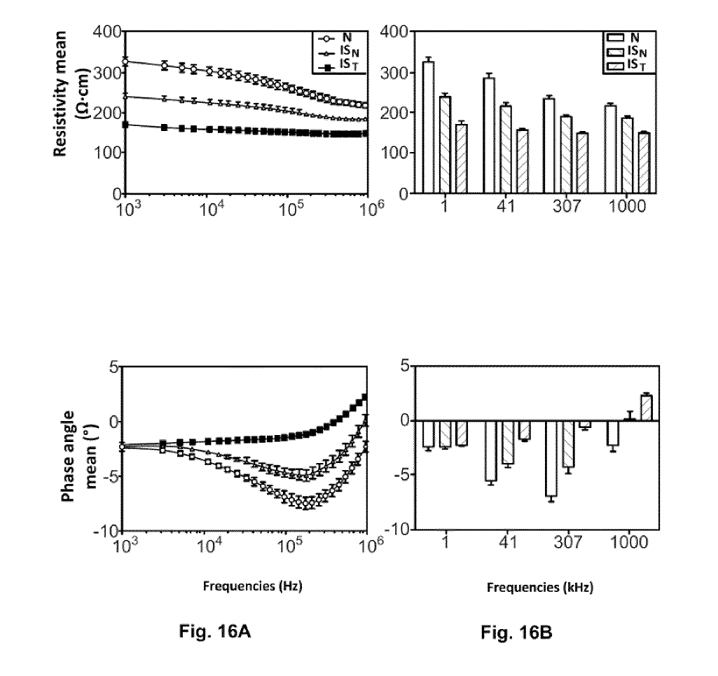

FIG. 16A illustrates the mean value of resistivity (top) and phase angle (bottom) vs. the excitation frequency for three different tissues: healthy tissue (N), non-transmural and transmural infarcted scar (IS_{N} and IS_{T}, respectively). FIG. 16B illustrates the mean values of resistivity (top) and phase angle (bottom) at 4 selected frequencies (1, 41, 307 and 1000 kHz) for the three tissues (***=p<0,001; **=p<0,01).

As shown in FIGS. 16A and 16B, the mean impedance values during the cardiac cycle of the normal myocardium declines significantly at increasing current frequencies (N: 285.5±10.4 vs. IS_{N}: 216.4±7.3 vs. IS_{T}: 155.5±6 a cm, at 41 kHz; p<0.001) and the phase angle shows a negative relaxation that reaches a maximum at about 307 kHz (N: −5.6±0.4 vs. IS_{N}: −4±0.4 vs. IS_{T}: −1.7±0.2°, at 41 kHz; p<0.001). In contrast, the infarct scar shows lower resistivity values and less marked phase angle deviation at all frequencies.

Moreover, the curves of frequency dependence of resistivity and phase angle depict a progressive attenuation towards the sites as the recording site moves to areas with transmural necrosis. The current frequencies that better differentiate the transmural and non-transmural scar are 1 kHz, 41 kHz and 307 kHz for resistivity and 41 kHz, 307 kHz and 1 MHz for the phase angle.

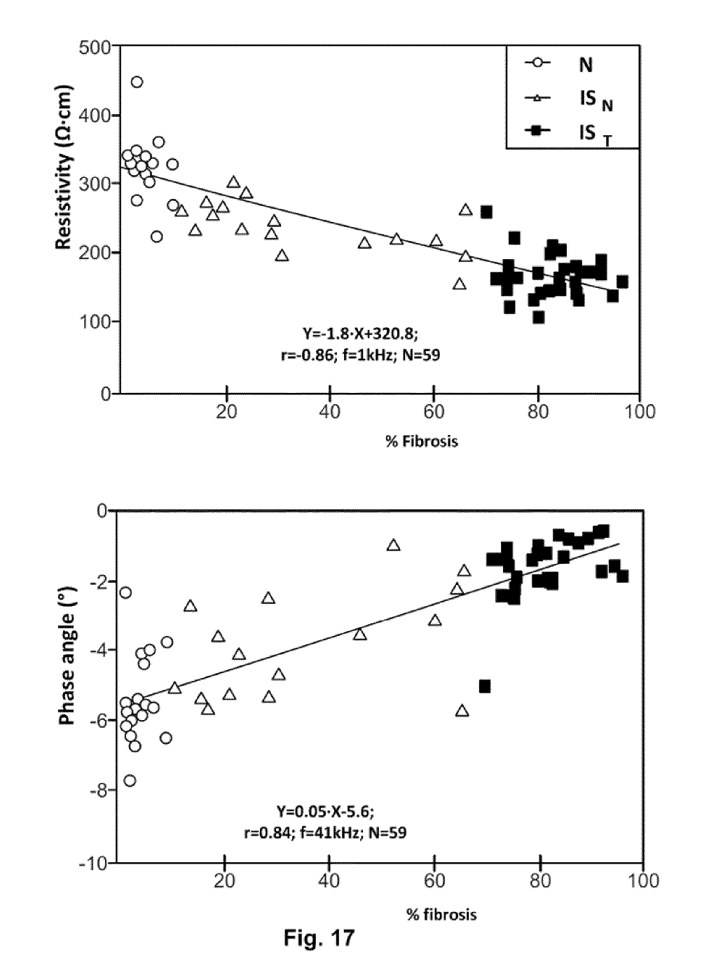

FIG. 17 illustrates a linear correlation between myocardial resistivity (upper-left)/phase angle (lower-left) and the percentage of fibrosis. White filled circles correspond to samples with <10% fibrosis, gray triangles to samples with 10% to 70% of fibrosis and black squares to samples with fibrosis higher than 70%. As illustrated in the figure, tissue samples analyzed taken at the sites with corresponding impedance measurements show that myocardial resistivity correlates negatively with the degree of fibrosis (r=-0.86 at f=1kHz, p<0.001) whereas a positive correlation is observed between phase angle and the fibrotic content (r=0.84 at f=41kHz, p<0.001). The best correlation coefficient is obtained at 1 kHz for myocardial resistivity and 41 kHz for phase angle (Table 1).

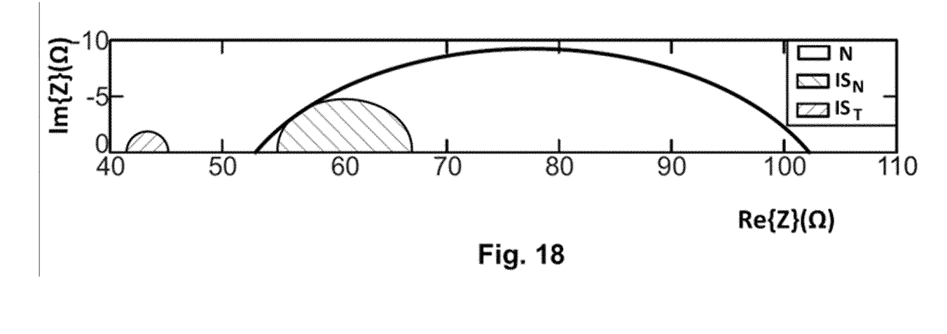

FIG. 18 illustrates the comparative pattern of the Cole impedance model obtained in normal (N: white), non-transmural scar (IS_{N}: grey) and infarcted transmural scar (IS_{T}: black). As the recording site moved from the normal region towards the non-transmural and transmural infarcted area, the Cole arc showed two main changes: a progressive decrease of the area under the curve and a left and down shift towards lower resistivity values (real and imaginary components, respectively). Table 2 summarizes the mean values of the Cole parameters (R₀, R_{inf}, α and f_{c}) in 59 tissue samples obtained from the 3 index myocardial regions.

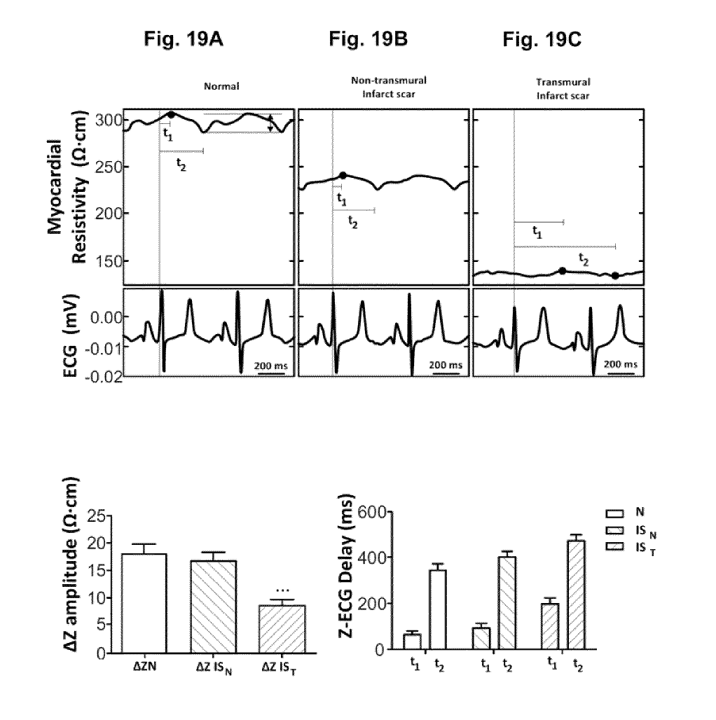

As illustrated in FIG. 19A, myocardial tissue electrical resistivity in the normal myocardium depicts a phasic pattern during the cardiac cycle: a peak shortly after the R wave and a minimum value appear in the region of the T wave. As illustrated in FIG. 19B, the non-transmural infarcted region depicts a pattern similar to normal myocardium. By contrast, as illustrated in FIG. 19C the transmural necrotic region shows a marked attenuation of the magnitude of the resistivity changes with a delayed occurrence of the maximal and minimal resistivity values.

Univariate analysis showed that the impedance parameters that detect differences between the three tissue categories are the mean value of the resistivity and the phase angle during the cardiac cycle at 41 kHz. Using multinomial logistic regression it has been found that the resistivity has the highest probability to correctly classify between healthy tissue, non-transmural and transmural infarct scar.

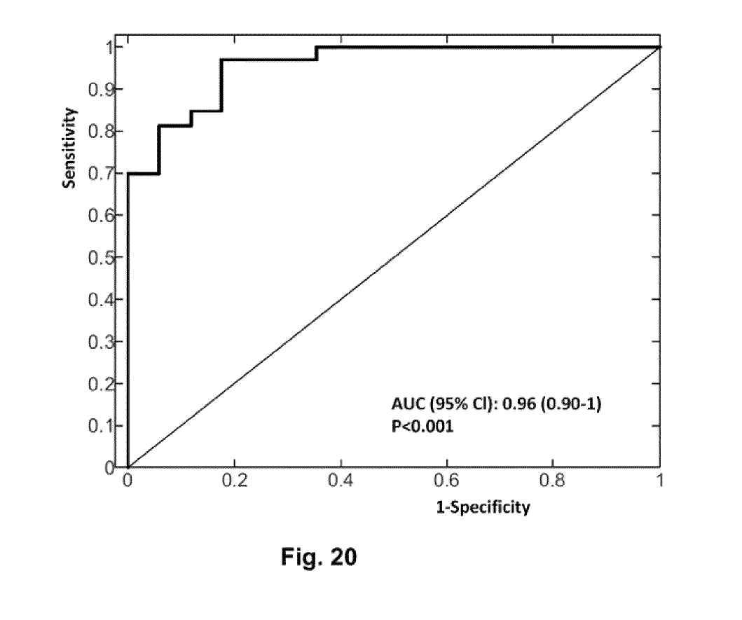

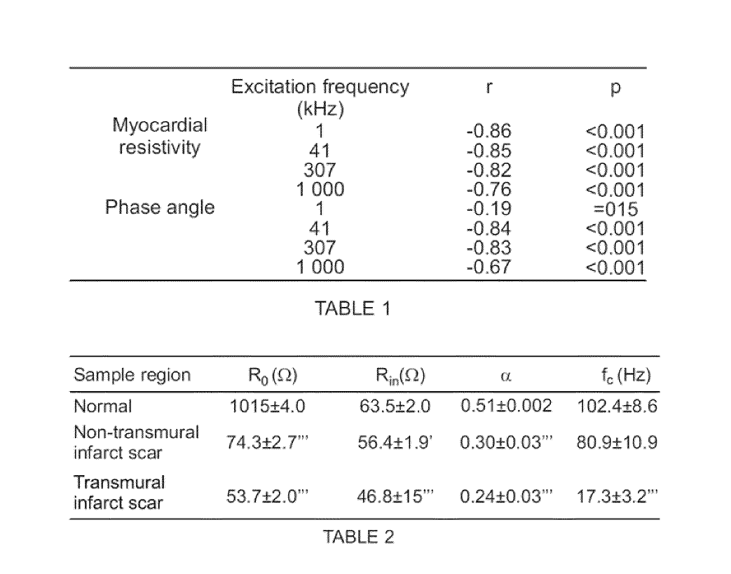

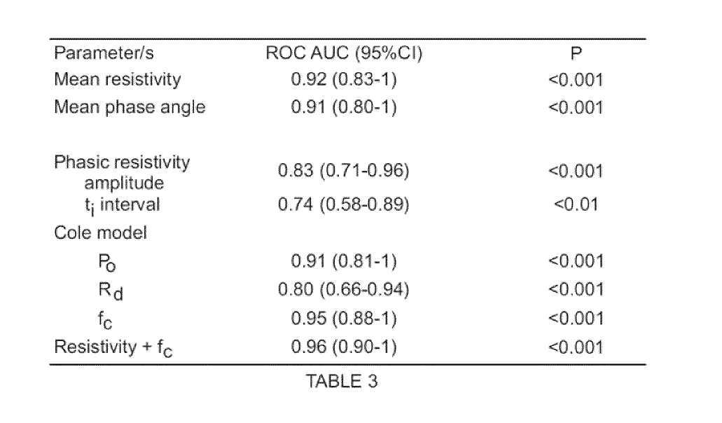

To further assess the predictive ability of different impedance parameters to discriminate between scar tissues with transmural and non-transmural affectation, the analysis of ROC (receiver operating characteristic) curves were used. Table 3 shows the accuracy of the ROC curve assessed by the area under the ROC curve (AUC) for all the different studied variables with its statistical significance levels. Resistivity, phase angle, resistivity amplitude, delay t₁, R₀, R_{inf}, and f_{c} exhibit good values of AUC with significant p-values, being f_{c} the best parameter. As illustrated in FIG. 20 which is a multiparametric analysis showing that a combination of resistivity and f_{c} improve the predictive ability to discriminate between both fibrotic tissues.

Although only a number of examples have been disclosed herein, other alternatives, modifications, uses and/or equivalents thereof are possible. Furthermore, all possible combinations of the described examples are also covered. Thus, the scope of the present disclosure and claims should not be limited by particular examples, but should be determined only by a fair reading of the claims that follow.

Further, although the examples described with reference to the drawings include computing apparatus/systems and processes performed in computing apparatus/systems, the invention also extends to computer programs, particularly computer programs on or in a carrier, adapted for putting the system into practice.

1. A method of assessing a cardiac tissue, comprising:
selecting an area of interest of the cardiac tissue;
identifying one or more measurement locations in the selected area of interest;
placing an electrocatheter probe at the one or more measurement locations;
providing a broadband spectrum signal to the one or more measurement locations using the electrocatheter probe;
identifying a diastolic phase of the a cardiac cycle;
measuring impedance of the cardiac tissue during the identified diastolic phase to obtain a diastolic impedance measurement;
identifying a systolic phase of the cardiac cycle;
measuring impedance of the cardiac tissue during the identified systolic phase to obtain a systolic impedance measurement;
assessing said cardiac tissue based on said diastolic and systolic impedance measurements.

2. (canceled)

3. (canceled)

4. A method according to claim 1, one or both of the diastolic and systolic impedance measurements being obtained either
between two electrocatheters, a first one of said two electrocatheters configured to be placed at a first location of the cardiac tissue and a second one of said two electrocatheters being configured to be placed at another a second location of the cardiac tissue or at a position on a body associated with the cardiac tissue, or between an electrocatheter configured to be placed at a location of the cardiac tissue and a selected electrode placed at a location on the body associated with the cardiac tissue.

5. (canceled)

6. A method according to claim 1 further comprising, classifying at least one of the one or more measurement locations as either a normal region or an infarct scar region in response to one or both of said diastolic and systolic impedance measurements.

7. A method according to claim 6, further comprising:
identifying resistivity and phase angle parameters of the impedance measurements and
wherein the classifying comprising correlating one or both of the identified resistivity and the phase angle components with a fibrosis percentage.

8. A method according to claim 7, the broadband signal compriseing either
multiple current frequencies between 1 kHz and 1MHz, or
one or more frequencies selected from a 1 kHz, 41 kHz, 307 kHz and 1 MHz frequencies.

9. (canceled)

10. A method according to claim 7, further comprising identifying a transmurality percentage of the cardiac tissue.

11. (canceled)

12. (canceled)

13. A method according to claim 1, further comprising recording a number of spectra along the cardiac cycle that allow reconstructing time-domain signals at different and simultaneous frequencies and using indicators of the signals shape to assess the cardiac tissue.

14. (canceled)

15. (canceled)

16. A method according to claim 1, the measuring impedance comprising measuring one or more intrinsic variables of impedance or measuring admittance and calculating impedance thereafter as a function of the measurements.

17. A method according to claim 16, the measuring impedance further comprising taking into account one or more values of and absolute or relative changes of the magnitude or phase angle or alternative representations thereof as real and imaginary parts, and the model parameters after fitting any of the variables spectrum to a mathematical model, their time course or their values in selected points of the cardiac cycle.

18. A method according to claim, the assessing of the tissue comprising deriving a state of the tissue either from
pathology-dependent delays between the reference signals (arterial or left ventricular pressure, ECG) and impedance related signals; or from
pathology-dependent shape and/or area of a figure resulting from a representation of an impedance-related variable at one or several frequencies and ventricle pressure.

19. (canceled)

20. A device, configured to
select an area of interest of a cardiac tissue;
identify one or more measurement locations in the selected area of interest;
an electrocatheter probe at the one or more measurement locations;
provide a broadband signal to the one or more measurement locations using the electrocatheter probe;
identify a diastolic phase of the a cardiac cycle;
measure impedance of the cardiac tissue during the identified diastolic phase to obtain a diastolic impedance measurement;
identify a systolic phase of the cardiac cycle;
measure impedance of the cardiac tissue during the identified systolic phase to obtain a systolic impedance measurement;
assessing said cardiac tissue based on said diastolic and systolic impedance measurements.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A device, comprising:
an arbitrary waveform generator (AWG) configured to deliver one or more broadband frequency current signals having an amplitude and a duration lasting over a time period associated with one or more cardiac cycles;
a multielectrode probe, coupled to the AWG, configured to apply the broadband frequency current signal in vivo to a cardiac tissue and measure impedance of the cardiac tissue thereby generating impedance measurements;
an acquisition module (AM) to generate a recording, the AM comprising:
an electrocardiograph (ECG) recorder;
a blood pressure recorder;
a controller, coupled to the AWG and to the AM and configured to
receive the impedance measurements during the duration of the broadband signal;
receive the recording of the acquisition module during the duration of the broadband signal;
identify one or both of a systolic or diastolic phase of a cardiac cycle;
correlate the impedance measurements with the identified phase of the cardiac cycle;
identify a state of the cardiac tissue as transmural or non-transmural depending on said correlation.

41. A device according to claim 40, the multielectrode probe comprising a transcatheter probe.

42. A device according to claim 41, the transcatheter probe comprising:
a tip electrode arranged at or near a tip of the transcatheter probe, along with one or more of:
one or more ring electrodes arranged around the transcatheter probe and at one or more distances from the tip electrode, respectively; and/or
an array of four electrodes arranged at a side or at a tip of the transcatheter probe; and/or
two electrocatheters configured to be placed at different locations of the cardiac tissue; and/or
an ablation electrode at a tip of the transcatheter probe.

43. (canceled)

44. (canceled)

45. (canceled)

46. A device according to claim 42, further comprising a mapping tool to identify an ablation zone comprising all identified transmural and non-transmural ischemic tissue in a region of interest.

47. A device according to claim 46, the multielectrode probe being further configured to apply an ablation current to the ablation electrode to ablate the identified ablation zone.

48. A device according to any of claims 40, the controller comprising a front-end to select and adapt the signals to and from the electrodes and a processor to synchronize the generation and the acquisition, control the acquisition sequence and send the results to an external processing and visualization system, the external processing and visualization system being configured for acquiring time-frequency information of quasi-static electrical impedance spectra in a given location of the myocardium and in several temporal points of the cardiac cycle.

49. A device according to claim 48, the front-end being configured to be adaptable to several electrode configurations of 2, 3 or 4 electrode configurations and switch between them.

50. (canceled)

51. (canceled)

52. A device according to claim 40, the controller being configured to one or more of:
identify the state of the cardiac tissue as a fibrosis state, and/or
generate a transmurality fibrosis map of the cardiac tissue as a function to the impedance measurements and the identified phase, and/or
be coupled to an ablation tool to provide an ablation map based on the transmurality fibrosis map in conjunction to the voltage map.

53. (canceled)

54. (canceled)

55. (canceled)

56. (canceled)

57. (canceled)

58. A system comprising:
a device according to claim 40,
an external processing apparatus, connectable to the device via a communication link, the external processor apparatus configured to run an application to determine a suitable waveform to be uploaded in the AWG, an acquisition strategy, and to apply algorithms to obtain values of tissue state estimators from time-frequency characteristics of the measured impedance signals.
